(19) United States
(12) Patent Application Publication
(10) Pub. No.: US 2023/0291111 A1
(43) Pub. Date: Sep. 14, 2023

(54) MULTI-MODAL ANTENNA

(71) Applicant: THE UNIVERSITY OF QUEENSLAND, St Lucia, Queensland (AU)

(72) Inventors: Feng LIU, St Lucia, Queensland (AU); Stuart CROZIER, St Lucia, Queensland (AU); Mingyan LI, St Lucia, Queensland (AU); Ewald WEBER, St Lucia, Queensland (AU); Aurélien DESTRUEL, St Lucia, Queensland (AU); Jin JIN, St Lucia, Queensland (AU)

(21) Appl. No.: 18/019,011

(22) PCT Filed: Aug. 3, 2021
(86) PCT No.: PCT/AU2021/050846

§ 371 Date: Jan. 31, 2023

(30) Foreign Application Priority Data
Aug. 4, 2020 (AU) 2020902725

(51) Int. Cl. H01Q 9/26; H01Q 21/00; H01Q 21/06; H01Q 21/20

(52) U.S. Cl. CPC H01Q 9/26; H01Q 21/0025; H01Q 21/062; H01Q 21/20

(57) ABSTRACT

A multi-modal antenna for use in magnetic resonance applications, the multi-modal antenna including an elongate first conductive element, an elongate second conductive element at least partially aligned with and spaced from the first conductive element and a dielectric material at least partially separating the first and second conducting elements so that the first and second conductive elements are electromagnetically coupled and/or electrically connected, and wherein at least one of the first and second conducting elements are configured to be electromagnetically coupled and/or electrically connected to an RF system so that the multi-modal antenna can at least one of transmit and receive RF electromagnetic signals for performing magnetic resonance imaging or spectroscopy.

### BACKGROUND OF THE INVENTION

The present invention relates to a radiofrequency (RF) multi-modal antenna for use in magnetic resonance applications, and in one particular example an Integrated Multi-modal Antenna with coupled Radiating Structures (I-MARS).

### DESCRIPTION OF THE PRIOR ART

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Ultra-high-field (UHF) whole body Magnetic Resonance Imaging (MRI) and Magnetic Resonance Spectroscopy (MRS) systems with a main magnetic field strength of 7 Tesla or higher, have seen significant development. Imaging and spectroscopy of certain regions of the human body, such as extremity and head, using UHF systems has demonstrated superior quality and sensitivity in comparison to using scanners of lower field strengths. However, the advantage of UHF systems for a large body section such as a hip joint or the abdomen, or deep anatomies such as prostate and heart, has not been well demonstrated mainly due to the lack of suitable radiofrequency (RF) coils.

For UHF body MRI/MRS applications, the RF transmit magnetic fields (B1+) may exhibit severe inhomogeneity. In a lower field MRI/MRS system, the RF transmission is often performed by a volume RF coil, typically of a cylindrical shape and located inside and adjacent to the inner wall of the scanner bore. However, such coils are associated with non-uniform B1+ fields at UHF. In MRI/MRS, B1+ is responsible for exciting the protons in the imaging region; after the B1+ is removed the excited protons go through a relaxation process while emitting the so-called “magnetic resonance (MR) signal”. It is based on this signal that images and spectra can be created for diagnosis and research purposes. Inhomogeneous B1+ is associated with spatially non-uniform excitation of protons, severely degrading the uniformity of the signal and therefore the clinical value of the MRI/MRS.

Parallel transmit systems (pTx) have been one of the most promising techniques developed to improve excitation profiles in UHF applications. pTx coils typically comprise an array of transmit elements distributed around the body section to be scanned, and RF amplifiers to independently drive individual coil elements. To achieve desired excitation profiles with the pTx system, numerical algorithms are then used to optimize the amplitude, phase and/or shape of the signal waveforms to drive the transmit elements. For example, constructive interferences of the individual B1+ fields can be used to provide sufficient excitation at a targeted scan region. In another example, the designed RF waveforms are combined with the MRI gradient systems to provide the so-called “spatially selective” pulses, with which the entire field of view or only a selective region can be excited with uniform intensity. As opposed to traditional coils, these techniques in combination pTx RF coils allow to control the coil efficiency and reduce the specific absorption rate (SAR), a measure of the RF energy absorbed by tissues.

Surface coil arrays that have both transmit and receive abilities, namely RF transmit-receive or transceive coils, are becoming popular because both RF transmission and reception systems are integrated to maximize the efficiency, instead of competing for space in close proximity to the region of interest. RF transceive coils offer improved power efficiency in transmit mode and better signal-to-noise ratio (SNR) in receive mode. Additional electronics are needed to allow the same RF coil elements to switch between the transmit and receive modes.

Conventionally, RF arrays use surface coil elements that are historically of loop shapes. In theory, a loop coil is equivalent to a magnetic dipole, ideally suited to produce magnetic fields perpendicular to the loop plane. In reception, changes in magnetic flux (produced by the excited nuclear magnetization in the imaged subjects) induce a current in the loop according to the Faraday's Law of induction. The resonance of a loop antenna is achieved by adjusting the inductance (size of the metallic loop) and capacitance (discrete or distributed form) of the circuit. At higher radio frequencies associated with UHF MRI/MRS, however, the transmit and receive magnetic field profiles of loop-shaped RF elements are less than ideal, encouraging the search for better RF elements.

Recently, dipole antennas as RF surface coil elements have become popular for UHF imaging applications. A dipole antenna typically consists of two identical conductive arms, symmetrically located with respect to the feeding/port. The resonance of a ½ wavelength dipole, as typically used in MRI applications, is achieved by creating standing waves of electrical currents oscillating between the two arms. It has been shown that the electric current pattern on a dipole antenna was more suited for UHF than their loop counterparts.

Regardless of the type of antenna, there are several important considerations when designing array elements for UHF applications. They include:
Criterion 1: High power efficiency. In transmission, the UHF RF transmission systems are typically limited in the power provided by the equipped RF power amplifiers. Therefore, the RF coil should be efficient to provide adequate excitation (B1+ magnitude) as demanded by the imaging or spectroscopy applications. When RF coils are used to receive MR signals, by virtue of the principle of reciprocity, high transmit efficiency is indicative of high receive sensitivity, which is important for high SNR when supporting electronics are properly designed and implemented.
Criterion 2: Low RF energy exposure. High RF energy deposition, measured by the SAR, may lead to temperature-induced damage in tissue. RF energy deposition in tissue is an important design criterion particularly for UHF because energy deposition increases quadratically with the field strength, and the local energy hot spots are highly influenced by RF coil design. In fact, global and local SAR are typically the limiting factors of practical UHF applications.
Criterion 3: Low coupling between channels. Due to the lack of a body coil, UHF MRI typically uses local transmit and receive coil arrays. Low coupling between transmit channels are essential to improve transmit efficiency and pTx capability; and low coupling between receive channels enhances receive SNR by reducing noise covariance.
Criterion 4: High stability with regard to imaging subjects and body parts. The local transmit and receive coil arrays are typically placed in close proximity to the region of interest, and therefore more sensitive to loading changes compared with large volume coils. The loading changes can be the results of different coil placements between scans and different body anatomies between patients. Conventional RF coils typically experience resonance frequency shifts and sub-optimal matching when loading conditions vary, which decrease transmit and receive efficiency.

Rapid development and uptake of dipole antenna multi-element array coils has occurred in the pursuit of obtaining an ideal current pattern that yields high efficiency (criterion 1), high element SNR (criterion 1) and low element SAR (criterion 2) for use at 7T, as described for example in Lattanzi R, Sodickson DK. “Ideal current patterns yielding optimal SNR and SAR in magnetic resonance imaging: computational methods and physical insights”. Magnetic Resonance in Medicine 2012; 68(1):286-304.

Recent designs aimed to shorten their physical length for practical application from the theoretical half-wavelength (approximately 48 cm long in air for 7T applications). These designs include “fractionated dipole antenna” (Raaijmakers A J E, Italiaander M, Voogt I J, Luijten P R, Hoogduin J M, Klomp D W J, van den Berg CAT. “The fractionated dipole antenna: A new antenna for body imaging at 7 Tesla”. Magnetic Resonance in Medicine 2016; 75(3):1366-1374.), a “single-side adapted dipole (SSAD)” (Raaijmakers AJE, Ipek O, Klomp D W J, Possanzini C, Harvey P R, Lagendijk J J W, van den Berg CAT. “Design of a Radiative Surface Coil Array Element at 7 T: The Single-Side Adapted Dipole Antenna”. Magnetic Resonance in Medicine 2011; 66(5):1488-1497) and hybrid loop-dipole (“loopole”) (Lakshmanan K, Cloos M, Lattanzi R, Sodickson D, Wiggins GC. “The loopole antenna: capturing magnetic and electric dipole fields with a single structure to improve transmit and receive performance”. 2014; Milan, Italy. p 397). These examples demonstrate considerable promises for 7T in vivo applications. However, these designs do not actively consider criteria 3 or 4. In fact, these existing element designs are sensitive to variations in loading conditions, and the decoupling between elements typically relies on having a large distance between elements, preventing high-density array designs and reducing imaging performance in certain Regions of Interest (ROI).

Although dipole current distribution may be suitable at 7T, conventional dipoles suffer from poor stability when the loading condition is varied (e.g., the position and/or electrical properties change among patients). The subsequent changes in the tuning and/or matching of the elements would significantly reduce their efficiency, degrade the image quality, and in extreme cases damage hardware. Similar issues are associated with conventional loop-shaped antennas. Alternative designs, such as shielded resonators or multi-layer resonators, have been proposed to achieve lower coupling (criterion 3) and higher loading stability compared to a conventional loop coil (criterion 4). Recently, a similar structure has been used to design a “self-isolated” loop coil. However, such technology has not been presented with RF dipole elements.

### SUMMARY OF THE PRESENT INVENTION

In one broad form the present invention seeks to provide a multi-modal antenna for use in magnetic resonance applications, the multi-modal antenna including: an elongate first conductive element; an elongate second conductive element at least partially aligned with and spaced from the first conductive element; and, a dielectric material at least partially separating the first and second conducting elements so that the first and second conductive elements are electromagnetically coupled and/or electrically connected, and wherein at least one of the first and second conducting elements are configured to be electromagnetically coupled and/or electrically connected to an RF system so that the multi-modal antenna can at least one of transmit and receive RF electromagnetic signals for performing magnetic resonance imaging or spectroscopy.

In one embodiment at least one of: the first and second conducting elements operate in one of: a transmission line mode; a dipole mode; and, a combination of a transmission line mode and a dipole mode; and, the dielectric layer and the first and second conductive elements form a transmission line.

In one embodiment the first conductive element is stimulated by the RF system and the second conductive element is stimulated by the first conductive element.

In one embodiment the first and second coupled conductive elements are stimulated by the MR signal from the subject.

In one embodiment the first and second conductive elements cooperate to define a closed-loop current including conductive currents passing along the first and second conductive elements and displacement currents passing through the dielectric material.

In one embodiment at least one of the conductive elements has a dipole configuration.

In one embodiment at least one of the conductive elements includes a slot or cut-out to define two arms, and wherein the RF system is electrically connected and/or electromagnetically coupled to each arm.

In one embodiment each conductive element at least one of: includes slots or cut-outs; has a length greater than a width; has a width greater than a thickness; is substantially laminar; is substantially planar; is at least partially flexible so that the multi-modal antenna can conform to a shape of a subject; is at least partially curved so that the multi-modal antenna can conform to a shape of a subject; includes an axial cross sectional shape that is at least one of: rectangular; circular; and, elliptical; has a paddle-shaped profile including one or more end portions wider or narrower than a mid-portion; has one or more meandering portions extending widthwise and lengthwise to increase an effective electrical length of the conductive element; includes multiple paddle stages; includes multiple paddle stages having different relative widths; and, includes multiple stages having different relative widths, and wherein a chamfer angle between stages can be adjusted.

In one embodiment the first and second conductive elements are interconnected via at least one of: lumped elements, additional conductive elements; and a direct connection.

In one embodiment the second conductive element at least one of: is smaller than the first conductive element; is shorter than the first conductive element; is narrower than the first conductive element; and, has a complementary profile to the first conductive element.

In one embodiment a spacing between the first and second conductive elements is at least one of: at least 0.1 mm; at least 1 mm; less than 10 mm; and, about 3 mm.

In one embodiment the first and second conductive elements are spaced at least one of: in a substantially parallel arrangement; and, asymmetrically.

In one embodiment the dielectric material is at least one of: is partially sandwiched between the first and second conductive elements; is provided in a layer; includes a number of layers of dielectric material; and, includes at least two different materials having different dielectric properties.

In one embodiment the multi-modal antenna includes: a dielectric layer; an outer conductive layer on at least one surface of the dielectric layer; and an inner conductive layer within the dielectric layer.

In one embodiment: the outer conductive layer includes the first conductive element; and, an inner conductive layer includes the second conductive element.

In one embodiment the dielectric material has a permittivity constant of at least one of: at least 1; less than 10; less than 35; less than 50; less than 100; less than 250; less than 500; less than 1000; and, about 3.5.

In one embodiment the antenna includes at least one further conductive element and/or at least one further dielectric structure.

In one embodiment the antenna includes at least one secondary element that modifies an electromagnetic response of the antenna.

In one embodiment the at least one secondary element includes at least one of: at least one secondary dielectric material; and, at least one secondary conductive element.

In one embodiment the at least one secondary element spans a cut-out in the first conductive element.

In one embodiment the multi-modal antenna is configured to minimise an electric field within the subject.

In one embodiment the multi-modal antenna includes a housing configured to maintain a desired spacing between the subject and the first and second conductive elements.

In one embodiment the housing includes a foam for engaging the subject, the foam having a defined thickness to maintain the desired spacing.

In one embodiment the RF system includes at least one of: a signal generator configured to generate RF signals that are applied to the antenna to generate the RF electromagnetic field; a detector that detects signals originating within the subject; and, a control system that causes the RF system to send control signals that can be used to control supporting electronics including at least one of: active detuning circuits; switching electronics; and, active switches.

In one embodiment active switching electronics are implemented into the multi-modal antenna to enable at least one of: active detuning to allow separate transmit and receive antenna operation modes; active on/off switching of different segments in conductive elements to allow control of current and field distributions; active changing of the resonant frequency; and, active changing of the effective electrical length of the multi-modal antenna.

In one broad form the present invention seeks to provide a multi-modal antenna array for use in magnetic resonance applications, the multi-modal antenna array including a plurality of RF antennas, each RF antenna including: an elongate first conductive element; an elongate second conductive element at least partially aligned with and spaced from the first conductive element; and, a dielectric material at least partially separating the first and second conducting elements, wherein the first and second conductive elements are electromagnetically coupled and/or electrically connected, and wherein at least one of the first and second conducting elements are configured to be electromagnetically coupled and/or electrically connected to a multi-modal system so that the RF antenna can at least one of transmit and receive RF electromagnetic signals for performing magnetic resonance imaging or spectroscopy.

In one embodiment the antenna array includes additional decoupling technique between the antennas in the array.

In one embodiment active detuning is implemented to allow separate transmit and receive antenna array configurations.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:

FIG. 1A is a schematic cross sectional side view of an example of a traditional dipole antenna;

FIG. 1B is a schematic cross sectional side view of a first example of a multi-modal antenna including first and second conductive elements;

FIG. 1C is a schematic cross sectional side view of a second example of a multi-modal antenna including first and second conductive elements;

FIG. 1D is a schematic cross sectional side view of a third example of a multi-modal antenna including first and second conductive elements;

FIG. 1E is a schematic cross sectional side view of an example of current patterns in the antenna FIG. 1A;

FIG. 1F is a schematic cross sectional side view of an example of current patterns in the antenna FIG. 1B;

FIG. 1G is a schematic cross sectional side view of an example of current patterns in the antenna FIG. 1C;

FIG. 1H is a schematic cross sectional side view of an example of current patterns in the antenna FIG. 1D;

FIG. 1I is an example of a 3D model of a phantom and the antenna of FIG. 1D, as well as the central axial slice of the corresponding B1+;

FIG. 1J is a schematic cross-sectional side view of an example of dimensions of the phantom of FIG. 1I;

FIG. 2A is a schematic plan view of a first conductive element including split (top) and single (bottom) meandered end portions;

FIG. 2B is a schematic sagittal cross-sectional side view of the antenna configuration of FIG. 1D modified by the inclusion of lumped elements;

FIG. 3A is a schematic plan view of an example of a comparative Fractionated 1 dipole configuration;

FIG. 3B is a schematic plan view of an example of a comparative Fractionated2 dipole configuration;

FIG. 3C is a schematic plan view of an example of a comparative single-side adapted dipole (SSAD) configuration;

FIG. 3D is a schematic plan view of an example of a straight multi-modal antenna configuration (I-MARS Straight);

FIG. 3E is a schematic plan view of an example of a meandering multi-modal antenna configuration (I-MARS Meander);

FIG. 3F is a schematic plan view of an example of a paddle multi-modal antenna configuration (I-MARS Paddle);

FIG. 4A is a graph illustrating example reflection coefficients of the different types of dipole elements and multi-modal antennas for a coil-phantom distance of 5 mm;

FIG. 4B is a graph illustrating example reflection coefficients of the different types of dipole elements and multi-modal antennas for a coil-phantom distance of 10 mm;

FIG. 4C is a graph illustrating example reflection coefficients of the different types of dipole elements and multi-modal antennas for a coil-phantom distance of 15 mm;

FIG. 4D is a graph illustrating example reflection coefficients of the different types of dipole elements and multi-modal antennas for a coil-phantom distance of 20 mm;

FIG. 5 is a graph illustrating example reflection coefficients of the different types of dipole elements and multi-modal antennas when changing the electrical properties of the phantom;

FIG. 6A is an image illustrating an example of B1+ magnitude in a central slice of the phantom of FIG. 1J, normalized to 1W of accepted power, produced by the Factionated1 dipole of FIG. 3A;

FIG. 6B is an image illustrating an example of B1+ magnitude in a central slice of the phantom of FIG. 1J, normalized to 1W of accepted power, produced by the Factionated2 dipole of FIG. 3B;

FIG. 6C is an image illustrating an example of B1+ magnitude in a central slice of the phantom of FIG. 1J, normalized to 1W of accepted power, produced by the SSAD of FIG. 3C;

FIG. 6D is an image illustrating an example of B1+ magnitude in a central slice of the phantom of FIG. 1J, normalized to 1W of accepted power, produced by the I-MARS Straight of FIG. 3D;

FIG. 6E is an image illustrating an example of B1+ magnitude in a central slice of the phantom of FIG. 1J, normalized to 1W of accepted power, produced by the I-MARS Meander of FIG. 3E;

FIG. 6F is an image illustrating an example of B1+ magnitude in a central slice of the phantom of FIG. 1J, normalized to 1W of accepted power, produced by the I-MARS Paddle of FIG. 3F;

FIG. 7A is a graph illustrating example B1+ magnitude along the dashed lines shown in FIGS. 6A to 6F, normalized to 1W of accepted power, in the absence of a shield;

FIG. 7B is a graph illustrating example B1+ magnitude along the dashed lines shown in FIGS. 6A to 6F, normalized to 1W of accepted power, with a shield spaced 5 mm from a rear of the dipole elements and multi-modal antennas;

FIG. 7C is a graph illustrating example B1+ magnitude along the dashed lines shown in FIGS. 6A to 6F, normalized to 1W of accepted power, with a shield spaced 10 mm from a rear of the dipole elements and multi-modal antennas;

FIG. 7D is a graph illustrating example B1+ magnitude along the dashed lines shown in FIGS. 6A to 6F, normalized to 1W of accepted power, with a shield spaced 15 mm from a rear of the dipole elements and multi-modal antennas;

FIG. 8A is a graph illustrating example B1+ magnitude along the dashed lines shown in FIGS. 6A to 6F, normalized to the square root of the peak SAR10g, in the absence of a shield;

FIG. 8B is a graph illustrating example B1+ magnitude along the dashed lines shown in FIGS. 6A to 6F, normalized to the square root of the peak SAR10g, with a shield spaced 5 mm from a rear of the dipole elements and multi-modal antennas;

FIG. 8C is a graph illustrating example B1+ magnitude along the dashed lines shown in FIGS. 6A to 6F, normalized to the square root of the peak SAR10g, with a shield spaced 10 mm from a rear of the dipole elements and multi-modal antennas;

FIG. 8D is a graph illustrating example B1+ magnitude along the dashed lines shown in FIGS. 6A to 6F, normalized to the square root of the peak SAR10g, with a shield spaced 15 mm from a rear of the dipole elements and multi-modal antennas;

FIG. 9A is an image of an example of decoupled I-MARS Meanders;

FIG. 9B is a close-up image of the example of decoupled I-MARS Meanders of FIG. 9A;

FIG. 9C is a graph illustrating an example of measured S-parameters of the decoupled I-MARS Meander pair, loaded with a torso, when connected with 220 nH inductors;

FIG. 9D is a graph illustrating an example of measured S-parameters of the I-MARS Meander pair, loaded with a phantom, with and without decoupling inductors;

FIG. 9E is a graph illustrating an example of simulated S-parameters of the I-MARS Meander pair, loaded with a phantom, with and without decoupling inductors;

FIG. 9F is an image illustrating an example of simulated B1+ magnitude of an I-MARS Meander, normalized to 1 W of accepted power;

FIG. 9G is an image illustrating an example of simulated B1+ magnitude of an I-MARS Meander, normalized to 1 W of accepted power adjacent another non-excited I-MARS Meander without decoupling;

FIG. 9H is an image illustrating an example of simulated B1+ magnitude of an I-MARS Meander, normalized to 1 W of accepted power adjacent another non-excited I-MARS Meander with decoupling;

FIG. 10A is an image of example manufactured I-MARS Paddle antennas in an open housing and a housing with a foam cover;

FIG. 10B is an image of an example of a 3D model of the I-MARS Paddle, showing its internal structure;

FIG. 10C is an image of an example of a 3D model of an assembled eight channel I-MARS Paddle coil antenna array;

FIG. 10D is an image of an example of an I-MARS Meander antenna;

FIG. 10E is an image of an example of an I-MARS array configured for unilateral shoulder imaging;

FIG. 11A is a schematic diagram of an example of an I-MARS Meander antenna array configured for unilateral hip imaging;

FIG. 11B is a schematic diagram of an example of an I-MARS Meander array configured for unilateral shoulder imaging;

FIG. 11C is a schematic diagram of an example of an I-MARS Meander array configured for bilateral hip imaging;

FIG. 11D is a schematic diagram of an example of an I-MARS Meander array configured for prostate imaging;

FIG. 11E is a schematic diagram of an example of an I-MARS Meander array configured for lumbar spine imaging;

FIG. 12A is a Magnetic Resonance image of an example of a unilateral 3D-DESS hip image captured using the configuration of FIG. 11A;

FIG. 12B is a Magnetic Resonance image of an example of a bilateral 3D-DESS hip image captured using the configuration of FIG. 11C;

FIG. 12C is a Magnetic Resonance image of an example of a unilateral shoulder image captured using the configuration of FIG. 11B;

FIG. 12D is a Magnetic Resonance image of an example of a T2w-TSE prostrate image captured using the configuration of FIG. 11D;

FIG. 12E is a Magnetic Resonance image of an example of a 3D-DESS lumbar image captured using the configuration of FIG. 11E using the posterior four coils only;

FIG. 12F is a Magnetic Resonance image of an example of a 3D-DESS lumbar image captured using the configuration of FIG. 11E;

FIG. 13A is a schematic side cross sectional view of an example of an I-MARS antenna with a secondary additional layer;

FIG. 13B is a schematic side cross sectional view of an example of a curved I-MARS antenna;

FIG. 13C is a schematic side cross sectional view of an I-MARS antenna when the inner conductive element is primarily coupled to the RF system;

FIG. 13D is a schematic cross-sectional view of an I-MARS antenna, including a second and a third dielectric material having different properties, distributed along the long axis of the element;

FIG. 13E is a schematic axial cross-sectional view of I-MARS antenna of FIG. 13B;

FIG. 13F is a schematic axial cross-sectional view of an example of an I-MARS antenna with two slots in a front conductive element;

FIG. 13G is a schematic axial cross-sectional view of an example of the I-MARS antenna of FIG. 1D with layers of dielectric of different electrical properties;

FIG. 13H is a schematic axial cross-sectional view of an example of the I-MARS antenna of FIG. 1D with asymmetric placement of an inner conductive element;

FIG. 13I is a schematic axial cross-sectional view of an example of the I-MARS antenna of FIG. 1D with an asymmetric outer geometry;

FIG. 13J is a schematic axial cross-sectional view of an example of the I-MARS antenna of FIG. 1D with a sloped placement of an inner conductive element;

FIG. 13K is a schematic axial cross-sectional view of an example of the I-MARS antenna of FIG. 1D with a stepped inner conductive element;

FIG. 14A is a schematic diagram of an I-MARS Paddle showing a first example internal structure;

FIG. 14B is a schematic diagram of an I-MARS Paddle showing a second example internal structure; and,

FIG. 14C is a schematic diagram of an I-MARS Paddle showing a third example internal structure.

### DETAILED DESCRIPTION OF THE PREFERRED EMBODIMENTS

An example of a multi-modal antenna for use in magnetic resonance applications, such as magnetic resonance imaging and/or spectroscopy will now be described.

In this example, the multi-modal antenna includes an elongate first conductive element and an elongate second conductive element at least partially aligned with and spaced from the first conductive element. A dielectric material is provided that at least partially separates the first and second conducting elements so that the first and second conductive elements are electromagnetically coupled and/or electrically connected. In use, one of the first or second conducting elements is configured to be electromagnetically coupled and/or electrically connected to an RF system so that the multi-modal antenna can at least one of transmit and receive RF electromagnetic signals for performing magnetic resonance imaging or spectroscopy.

In this configuration, one of the conductive elements is stimulated by the RF system, whilst the other conductive element is stimulated by electromagnetic fields generated by the stimulated conductive element. Thus, in one example, the first conductive element can be primarily stimulated by RF system, either directly via an electrical connection, or indirectly, for example via an inductive connection, and then the second conductive element is stimulated by the first conductive element, but it will be appreciated that reversed configurations could be implemented, in which the second conductive element is primarily stimulated. Additionally, and/or alternatively, when operating in receive mode, the first and second coupled conductive elements are stimulated by the MR signal originating within the subject.

In either case, the first and second conductive elements cooperate to define a closed-loop current including conductive currents passing along the first and second conductive elements and displacement currents passing through the dielectric material.

The above-described configurations result in a number of improved antenna characteristics. For example, the configuration minimises the external electric field that is generated, whilst maintaining a high external magnetic field, allowing the RF antenna to effectively stimulate the subject for magnetic resonance applications, whilst maintaining a high power efficiency, and low RF energy exposure. This further reduces coupling between different multi-modal antennas, whilst also providing high stability with regard to imaging subjects and body parts. Accordingly, it is apparent that the new antenna configurations can meet the criteria discussed above and represent a significant advancement over traditional arrangements.

A number of further features will now be described.

Typically the first and second conducting elements operate in a transmission line mode, a dipole mode, and more typically a combination thereof. In one specific example, the dielectric layer and the first and second conductive elements form a transmission line.

In one example, the first and/or second conductive element has a dipole configuration, and can include one or more slots or cut-outs. In one example, the slots or cut-outs define two arms, with the RF system being electrically connected to each arm, although it will be appreciated that slots or cut-outs can be provided in either of the first or second conductive elements, to adjust electrical properties as desired.

Each conductive element typically has a length greater than a width and a width greater than a thickness. The relative geometry (incl length, width and thickness) is adjusted to achieve optimal performance considering the wavelength of the applied signals. However, the length is typically in the region of 100 mm to 500 mm, 360 mm to 400 mm and more typically in the region of 376 mm to 380 mm, excluding shorter variations such as with meanders, paddle or lumped elements, as will be described in more detail below. The width is typically in the region of 10 mm to 25 mm and more typically about 18 mm, whilst the thickness is of the order of less than a few mm. Thus, it will be appreciated that the conductive elements are typically a thin substantially laminar body, and optionally, substantially planar, although the conductive elements may be curved and/or flexible so that the multi-modal antenna can more easily conform to a shape of a subject. For example, the multi-modal antenna could include flexible conductive elements embedded in a fluidic or otherwise deformable dielectric material. The conductive elements are typically made of copper or other similar materials, or a combination of multiple conductive materials.

In axial cross-section, the conductive elements typically have a rectangular shape, although this is not essential and other arrangements can be used, including, but not limited to circular, square and/or elliptical shapes.

In one example, the antenna has a paddle-shaped profile, including one or more end portions wider or narrower than a mid-portion or could include one or more meandering portions extending widthwise and lengthwise. The paddle-shape profiles could include multiple stages, which can have different relative widths, and may include chamfer regions where the stages join, with a chamfer angle being adjusted to obtain desired characteristics. These different arrangements, including the stages, paddle-shaped profile and meandering portions, act to more favourably redistribute electrical current density of the antenna structure for higher external magnetic fields generation and lower maximum local electrical energy in the subject, and/or increasing an effective electrical length of the antenna structure and reducing a physical length. These configurations can assist in making the antenna configuration more suitable for use in clinical or other environments, whilst maintaining the effectiveness of the antennas. A reduction in the physical length of the antenna can alternatively, or additionally, be achieved using lumped elements that interconnect the first and second conductive elements, which also provides the ability to adapt the distribution of electrical current. Current distribution could additionally and/or alternatively be achieved using additional conductive elements and/or dielectric elements and/or direct connections.

In one example, the second conductive element is smaller than the first conductive element, and could for example be shorter and/or narrower than the first conductive element, which can assist if the second conductive element is wholly embedded within the antenna, as will be described in more detail below. The second conductive element may or may not also have a similar profile to the first conductive element. In the former scenario, the conductive elements have substantially the same shape. In either case, the characteristics of the antenna are predominantly defined by the overlapping shape between the conductive elements, and the distribution of dielectric material between and/or around them, so the conductive elements could have significantly different shapes, with characteristics of the antenna being governed by the region of overlap of the conductive elements.

Typically a spacing between the first and second conductive elements is at least on 0.1 mm, at least 1 mm, typically less than 10 mm or more typically about 3 mm, although it will be appreciated that other spacings could be used depending on the preferred implementation, the intended use, the dimensions of the conductive elements, and the nature of the dielectric material. The conductive elements are typically in a substantially parallel arrangement, although this is not essential and other arrangements, such as asymmetrically spacing, relatively angling of the first and second conductive elements, or the like, could be used, depending on the characteristics of the antenna that are desired for the particular magnetic resonance application.

In one example, the dielectric material is partially sandwiched between the first and second conductive elements and may be provided in a layer, with conductive elements provided on one or more sides of, and optionally embedded within the layer. The dielectric material may also include two or more different materials having different dielectric constants, and in one example, can include two or more layers of dielectric material. The dielectric material has a permittivity constant of at least 1, less than 10, less than 35, less than 50, less than 100, less than 250, less than 500, less than 1000, or about 3.5, although different values could be used depending on the preferred implementation, the desired thickness of the dielectric layer, or the like.

In one configuration, the multi-modal antenna includes a dielectric layer, an outer conductive layer on at least one surface, and optionally extending paritally or completely around the exterior surfaces of the dielectric layer, with an inner conductive layer within the dielectric layer. In this example, the outer conductive layer can include the first (active) conductive element, whilst the inner conductive layer includes the second (passive) conductive element, although this is not essential and reversed arrangements could be used, with the internal conductive element being the active element.

In one embodiment the antenna includes at least one further conductive element and/or dielectric structure, and so for example, the antenna may include multiple second conductive elements spaced from the first conductive element, or could include third conductive elements spaced from the first and second conductive elements, thereby further helping ensure a desired distribution of currents within the antenna and/or fields within the subjects.

In one example, the antenna includes a secondary element that modifies an electromagnetic response of the antenna. This could include a secondary dielectric material and/or a secondary conductive element, and in one example spans a cut-out in the first or second conductive element, which can modify coupling between arms in the active dipole, and/or modify the magnetic and electric field distribution within the subject.

In general, the antenna is provided in a housing, optionally containing the first and second conductive elements, which is configured to maintain the desired spacing between the subject and the first and second conductive elements and may include a foam for engaging the subject, with the foam having a defined thickness to maintain the desired spacing.

As mentioned above, the multi-modal antenna is typically coupled to an RF system, which in one example can form part of a magnetic resonance apparatus configured to perform magnetic resonance imaging or spectroscopy. The RF system can include a signal generator configured to generate RF signals that are applied to the antenna to generate the RF electromagnetic field and may also include a detector that detects signals from the subject and/or a control system that causes the RF system to send control signals that can be used to control supporting electronics, such as active detuning circuits, switching electronics and/or active switches. Such active switching electronics can be implemented into the multi-modal antenna to enable at least one of: active detuning to allow separate transmit and receive antenna operation modes; active on/off switching of different segments in conductive elements to allow control of current and field distributions; active changing of the resonant frequency; and, active changing of the effective electrical length of the multi-modal antenna.

Whilst the antennas could be used separately, more typically a number of antennas are part of an antenna array. In this instance, properties of the antennas, in particular its multi-modal characteristics, can help reduce coupling between the individual antennas in the array. However, this can be further enhanced through the use of additional decoupling techniques, for example by connecting conductive elements in different antennas using inductive components.

Whilst the individual antennas and the antenna arrays can be used in transceive mode, active detuning circuits could be added to any of the individual antennas and antenna-elements in an array to enable additional transmit-only or receive-only modes. This is typically achieved by implementing electronically controlled switches, for example PIN diodes or other switching devices.

### SPECIFIC EXAMPLES

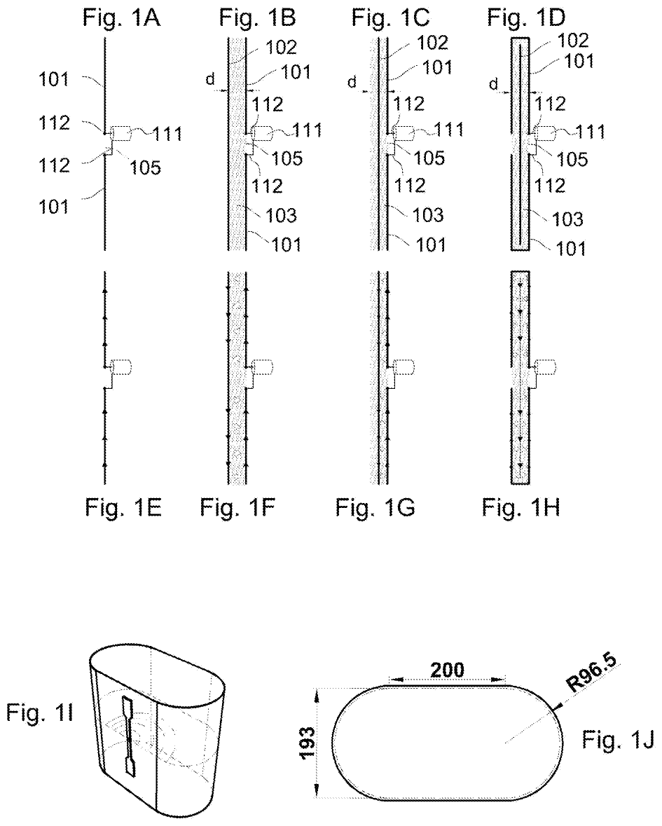

An example of a conventional dipole antenna is shown in FIG. 1A.

The dipole antenna is typically made of two arms 101 of conducting material, such as copper, with a slot 105 for RF signal feeding/receiving, which is typically achieved using a transmission line 111 connected to the arms 101, via connectors 112, although this could alternatively be achieved using indirect connections, such as via inductive coupling or the like. The transmission line 111 is typically connected to an RF system, such as a signal generator and/or sensor (not shown). Lumped elements may be used for tuning and matching purposes. In their simplest form, dipole antennas are designed to have an electrical length that approximates the half-wavelength of the transmitted or received signal. For the purpose of explanation, this dipole antenna will be referred to in the following study as a “Configuration A”, has a length of 380 mm and width of 22 mm.

Examples of multi-modal antenna configurations will now be described with reference to FIGS. 1B to 1D.

The first example multi-modal antenna configuration shown in FIG. 1B includes a first conductive element in the form of an actively excited dipole having two arms 101 separated by a slot 105. RF signal feeding/receiving is achieved using a transmission line 111 connected to the arms 101, via connectors 112. A second conductive element is provided in the form of a continuous passive conductor 102 separated from the first conductive element by a dielectric substrate 103 having a thickness d. For the purpose of explanation, this antenna configuration will be referred to in the following study as a “Configuration B”, and has a length and width similar to that of Configuration A.

A second example multi-modal antenna configuration is shown in FIG. 1C. This has a similar design to Configuration B, albeit with the second passive conductive element 102 being embedded within the dielectric 103 at a distance d/2 from the actively excited first conductive element formed by the dipole 101. For the purpose of explanation, this antenna configuration will be referred to in the following study as a “Configuration C”, and has a length and width similar to that of Configuration A.

A third example multi-modal antenna configuration is shown in FIG. 1D. This has a similar design to Configuration C, albeit with the second passive conductive element 102 being shortened and fully embedded within the dielectric 103, and the first conductive element formed by the dipole 101 extended to cover all the surfaces of the dielectric 103, except for a slot 105 for driving, receiving, matching and tuning. For the purpose of explanation, this antenna configuration will be referred to in the following study as a “Configuration D”, and in this example, the antenna has a length and width similar to that of Configuration A, but the second passive conductive element is decreased in size, having a length=376 mm and width=18 mm.

### Performance

To investigate the performance of the above designs, electromagnetic simulations were performed on these configurations in software Sim4Life (ZMT, Zurich, Switzerland), when loaded with the phantom shown in FIGS. 1I and 1J (height=656 mm, εᵣ=55, σ=0.66 S/m), placed 10 mm from the front of each element.

Table 1 shows the Power and SAR_{10g} efficiency of Configurations A-D with different relative permittivities and dielectric thicknesses. Table 1 shows the transmit B₁ power efficiency as a measure of the peak B₁⁺ and the B₁⁺ at a depth of 5 cm, as well as the peak-spatial SAR_{10g} (psSAR_{10g}) and the B₁⁺SAR efficiency (ratio between the B₁⁺ and the square root of the SAR_{10g}), for all configurations. For the configurations B-D, the dielectric thickness is varied (d=1, 3, 5 or 10 mm); so is the relative permittivity (εᵣ=1, 3.5, 5, 10 or 35). An electrical conductivity of σ=0.0015 S/m was used. All results were normalized to 1W of accepted power.

TABLE 1

| | Peak B₁⁺ | B₁⁺_{5 cm} | psSAR_{10 g} | B₁⁺_{5 cm}/√SAR_{10 g} |
|---|---|---|---|---|
| | μT | μT | W/kg | μT/√(W/kg) |
| Configuration A | 1.7 | 0.38 | 1.9 | 0.28 |
| Configuration B | | | | |
| εᵣ = 1, d = 3 mm | 2.35 | 0.45 | 2.64 | 0.28 |
| εᵣ = 3.5, d = 3 mm | 1.41 | 0.29 | 0.88 | 0.31 |
| εᵣ = 10, d = 3 mm | 1.38 | 0.33 | 1.32 | 0.29 |
| εᵣ = 35, d = 3 mm | 1.86 | 0.29 | 1.12 | 0.28 |
| εᵣ = 3.5, d = 1 mm | 1.38 | 0.27 | 0.76 | 0.31 |
| εᵣ = 3.5, d = 5 mm | 1.42 | 0.29 | 0.88 | 0.31 |
| εᵣ = 3.5, d = 10 mm | 1.39 | 0.28 | 0.78 | 0.31 |
| εᵣ = 3.5, d = 3 mm* | 1.78 | 0.32 | 1.13 | 0.3 |
| Configuration C | | | | |
| εᵣ = 3.5, d = 3 mm | 1.29 | 0.28 | 0.83 | 0.31 |
| εᵣ = 10, d = 3 mm | 1.32 | 0.32 | 1.19 | 0.29 |
| εᵣ = 35, d = 3 mm | 1.66 | 0.27 | 0.98 | 0.28 |
| εᵣ = 3.5, d = 1 mm | 1.35 | 0.24 | 0.65 | 0.3 |
| εᵣ = 3.5, d = 5 mm | 1.27 | 0.29 | 0.89 | 0.31 |
| εᵣ = 3.5, d = 10 mm | 1.18 | 0.3 | 0.89 | 0.31 |
| εᵣ = 3.5, d = 3 mm* | 1.58 | 0.33 | 1.19 | 0.3 |
| Configuration D | | | | |
| εᵣ = 3.5, d = 1 mm | 1.67 | 0.29 | 1.04 | 0.29 |
| εᵣ = 3.5, d = 3 mm | 1.51 | 0.33 | 1.31 | 0.29 |
| εᵣ = 3.5, d = 5 mm | 1.44 | 0.34 | 1.39 | 0.29 |
| εᵣ = 3.5, d = 10 mm | 1.32 | 0.35 | 1.41 | 0.29 |

*The width of the conductor on the load side was changed to 12 mm

As summarized in Table 1, all those configurations have different characteristics in terms of providing power efficiency (B₁⁺/√Power) or SAR efficiency (B₁⁺_{5cm}/√SAR_{10g}) (Criteria 1 and 2). In general, increasing d of Configurations B-D improved the power efficiency at 5 cm while maintaining the SAR efficiency in most cases. Additionally, in Configurations B-D εᵣ=3.5-10 gave the best compromises between power and SAR efficiency.

### Distributions of Electrical Currents

FIGS. 1E-H show illustrations of the conductive (black arrows) and displacement currents (open arrows) for the Configurations A-D, respectively.

FIG. 1E shows that the current density on the two arms of the conventional dipole have identical magnitudes. In contrast, Configurations B-D are operating in a different fashion, with the passive conductors in the individual configurations behaving as passively excited dipoles, which are coupled with the actively excited dipoles. In this work, they are collectively referred to as integrated multi-modal antennas with coupled Radiating Structures or I-MARS, because of the ways in which they operate.

Configuration B represents configuration, in which the conductive currents on the two dipoles have similar magnitude, albeit opposite phase. In Configuration C, the conductive currents mostly reside on the active dipole. Configuration D is a design that is symmetrical in radial direction. In this case, conductive currents mostly reside on the inner, passive dipole.

There exist multiple current modes within the structures of Configurations B-D. Within each configuration, the transmission-line mode currents on the pair of dipoles, which are out of phase from each other, together with the displacement currents within the dielectric substrates, form a closed current loop. In this loop-mode of resonance, the antenna structure operates in transmission line mode, whilst the dielectric substrate acts mainly as distributed capacitance. It is noted that besides induction, the closed-loop current is partially responsible for the excitation of the passive dipole in each of the Configurations B-D. The transmission-line mode co-exists with the dipole mode of resonance on the dipole pairs, while the dipole mode currents are in phase on the conductor pairs. The co-existence of the two resonance modes is described by the term “multi-modal”. “Integrated” in ‘I-MARS’ simply refers to the fact that the active, passive dipoles and dielectric substrate in each configuration are acting as a complete resonance structure. In fact, when the two resonance modes are considered as a whole, there exists an excess of electrical current on the active-passive dipoles pair, causing a net current. This net current is in a similar magnitude to that of the conventional dipole (Configuration A).

### Stability of I-MARS Against Loading Changes and Coupling

To further investigate the effects of the different design features of I-MARS (passive dipole placement, size, dielectric properties) on their sensitivity to load changes and inter-element coupling, additional simulations were conducted. A common baseline of all configurations was first established by tuning the antennas of Configurations A-D to resonate at 297 MHz with S₁₁=−20 dB when the phantom was 10 mm from the front of each element. Simulations were repeated with the phantom 15 mm from the front of each element, without altering the matching and tuning circuits from the corresponding baseline simulations. The S₁₁ at 297 MHz was recorded, as well as the shift in resonant frequency. In yet another set of simulations, by introducing another antenna of the same design to the corresponding baseline simulations, two elements of each design were simulated with a center-to-center distance of 55 mm. Their S₁₂ was recorded in Table 2 for analysis, with Table 2 showing sensitivity to loading and inter-element coupling for different configurations.

TABLE 2

| | Phantom-coil distance | | |
|---|---|---|---|
| | increased by 5 mm | | |
| | | Resonant | 55 mm center |
| | | frequency/frequency | to center |
| | S₁₁ in dB | shift in MHz | S₁₂ in dB |
| Configuration A | −11.4 | 321.4/24.4 | −11.3 |
| (conv. d) | | | |
| Configuration B | −12.1 | 296.64/−0.36 | −8.7 |
| εᵣ = 1, d = 3 mm | | | |
| Configuration B | −14.7 | 294.5/−2.5 | −9.8 |
| εᵣ = 3.5, d = 3 mm | | | |
| Configuration B | −14.2 | 291.78/−5.22 | −7.8 |
| εᵣ = 3.5, d = 10 mm | | | |
| Configuration C | −10.1 | 294.01/−2.99 | −10.7 |
| εᵣ = 3.5, d = 3 mm | | | |
| Configuration C | −13.9 | 291.7/−5.3 | −8.4 |
| εᵣ = 3.5, d = 10 mm | | | |
| Configuration D | −13.4 | 294.5/−2.5 | −11.6 |
| εᵣ = 3.5, d = 3 mm | | | |
| Configuration D | −13 | 284.7/−12.3 | −9.8 |
| εᵣ = 3.5, d = 10 mm | | | |

According to Table 2, the εᵣ=3.5, d=3 mm variants of Configurations B-D perform better overall than the εᵣ=3.5, d=10 mm variants. The former with smaller dielectric thickness d had much smaller resonance frequency shift when the load changed, and had noticeably better Sit values between two like antennas. In fact, the resonance frequency shift of the Configurations B-D of the εᵣ=3.5, d=3 mm variants were an order of magnitude smaller than that of the Configuration A (conventional dipole). These advantages of the smaller dielectric thickness d=3 mm also outweigh the SAR efficiency (B₁⁺_{5cm}/√SAR_{10g}) provided by the larger d=10 mm, which is less than 3% as illustrated in Table 1. Among all the configurations and their variants, Configuration B with εᵣ=3.5, d=3 mm had the best S₁₁=−14.7 dB when the load was moved away; Configurations B and D with εᵣ=3.5, d=3 mm had the smallest frequency shift of −2.5 MHz; and Configuration D with εᵣ=3.5, d=3 mm had the best inter-element isolation of S₁₂=−11.6 dB with a center-to-center distance of 55 mm.

Summarizing the investigations so far, the I-MARS coils satisfy all the design criteria listed in the background. Similar to conventional dipole designs, the conductive currents of the I-MARS elements have a “dipole mode” current on the conductive materials mostly in the longitudinal direction, as shown in FIGS. 1F to 1H, hence providing ‘ideal’ current pattern and associated properties (Design Criteria 1 and 2). In contrast to the conventional dipole antenna, an additional closed-loop “transmission-line mode” current exists with the I-MARS. Namely, current flows between the external and internal conductors occur, while the current loop is completed by the displacement current within the dielectric material, as illustrated in FIGS. 1F to 1H. The combination of those two modes causes electric and magnetic coupling mechanisms which partly cancel each other, resulting in a lower inter-element coupling and reduced sensitivity to loading (Design Criteria 3). Furthermore, the distributed capacitance aims to prevent the concentration of high electric fields observed near discrete capacitors used in certain coil designs (Design Criteria 4), as well as improving coil stability (Criteria 3).

### Practical Considerations

There are several practical aspects to consider making I-MARS coils more suitable for in vivo applications. The tuning of the I-MARS coils is accomplished by designing the cross-sectional profile (widths of the inner and outer conductors and their relative ratios), the electrical properties of the dielectric substrates and the physical length of the coil elements. The length of the presented I-MARS configurations is 380 mm, making it impractical to use in some applications.

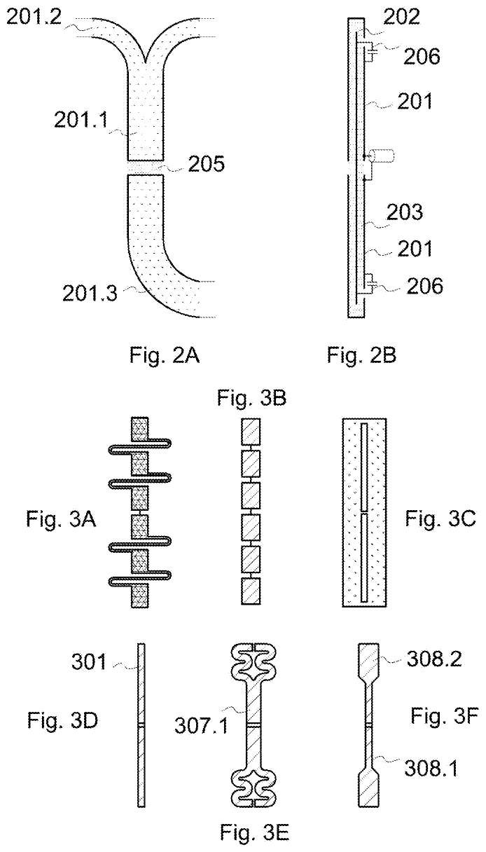

The optimal length, besides other geometric parameters, of I-MARS is determined on a case-by-case basis, while considering a number of metrics, such as, B₁ power efficiency, B₁SAR efficiency and stability (to be explained later). If increasing the electrical length is desirable, meanders or lumped elements can be introduced to achieve the same overall electrical length with a shorter physical length. Using Configuration D as an example, FIG. 2A shows the first conductive member, including a dipole having arms 201 with a central portion 201.1 and split meanders 201.2 or single meanders 201.3 in end portions. In such a configuration, the second conductor and dielectric substrate will substantially follow and align with the first conductor. In contrast, in the example of FIG. 2B, the antenna arrangement includes a first conductive dipole element 201, second passive conductive element 202 embedded within the dielectric 203, with additional lumped elements 206 interconnecting the dipole arms 201 and the passive conductive element 202. In the latter case, the potentially high electric fields introduced by the lumped elements, a typical drawback of using lumped elements to shorten conventional dipoles, are avoided because the lumped elements can be placed on the “feed side” of the dipole (opposite side of the patient) and the introduced electric fields can therefore be shielded by the element itself.

### Comparison Between 1-MARS and State-of-the-Art Dipoles:

Assisted with numerical electromagnetic simulations, the performance of the proposed I-MARS elements were compared with state-of-the-art dipole coil elements for UHF MRI/MRS.

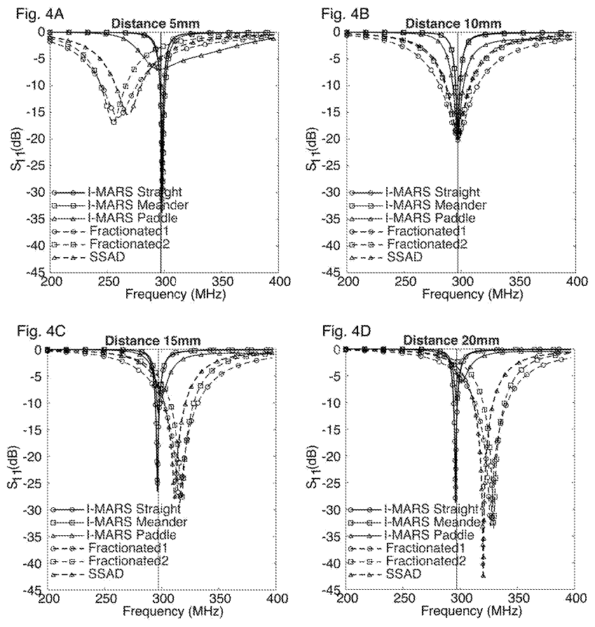

The fractionated antennas of FIGS. 3A and 3B, referred to respectively as Fractionated 1 and Fractionated2, have an overall length of 300 mm. To achieve self-resonance, both arms of the element were slotted with meanders shown in FIG. 3A or capacitors shown in FIG. 3B. The matched dipole, or SSAD arrangement shown in FIG. 3C includes of a conductive material (length/width=220/22 mm), placed on a block of matching material (length/width/thickness=240/55/22 mm, relative permittivity 36, and conductivity=6.12e-5 S/m).

This comparison includes I-MARS coils of three variations, all of which are based on Configuration D. The first variation, I-MARS Straight, as shown in FIG. 3D, has an active dipole 301 as outer conductive skin (length/width/thickness=380/22/1.28 mm), which encloses the dielectric substrate, and a central conductive plate as a passive dipole (length/width=380/18 mm). The outer skin has a slit in the middle of the structure on all four sides. RF feeding is provided on the slit on the “feed side” via a symmetrical matching network.

I-MARS Meander and I-MARS Paddle variations shown in FIGS. 3E and 3F are variations of I-MARS elements. For the I-MARS Meander in FIG. 3E, both the active dipole skin and passive dipole inner plate include a central portion 307.1 and end portions 307.2 that split and extend into the meanders. The I-MARS Paddle in FIG. 3F shows an additional variation to the I-MARS design, with a central portion 308.1 having a width of 10 mm and end portions 308.2 having a width of 30 mm. This design can redistribute the electrical current density along the element, which is desirable for the imaging of deep tissue such as hip joint and prostate. The dielectric material used in all I-MARS antennas was identical (relative permittivity of 3.5, and electric conductivity of 0.0015 S/m).

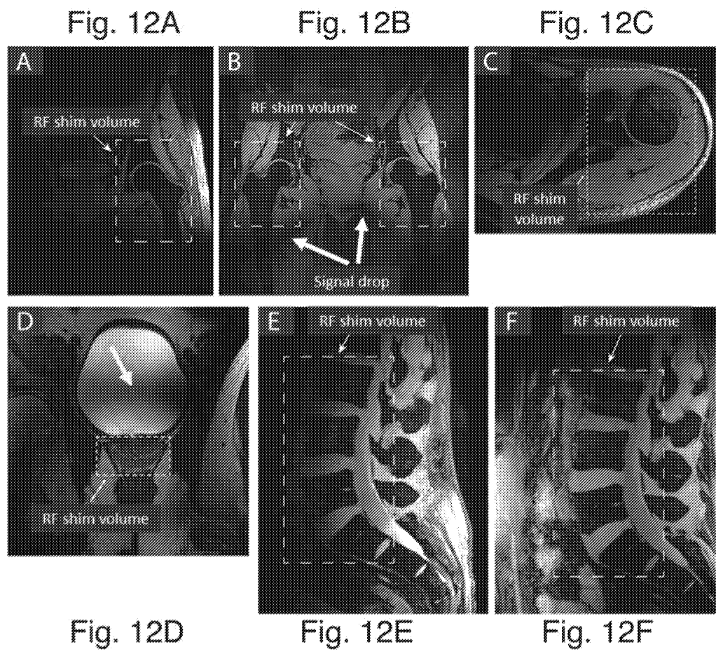

The centers of all the conventional and I-MARS elements were aligned with the center of the torso-shaped phantom shown in FIGS. 11 and 1J.

### Stability Against Loading Variations

To investigate the stability of all coil elements against loading changes, the scattering parameters were calculated when the body-mimicking phantom was located at different distances to the coil. A baseline simulation was established for each coil element at the original phantom position (10 mm away from the coil). The matching network and tuning lumped elements were optimized to achieve S₁₁=−20 dB at 297 MHz. The S₁₁ parameters were calculated again when the phantom was positioned 5, 15 and 20 mm away from the coils with the tuning and matching networks determined for the baseline simulation.

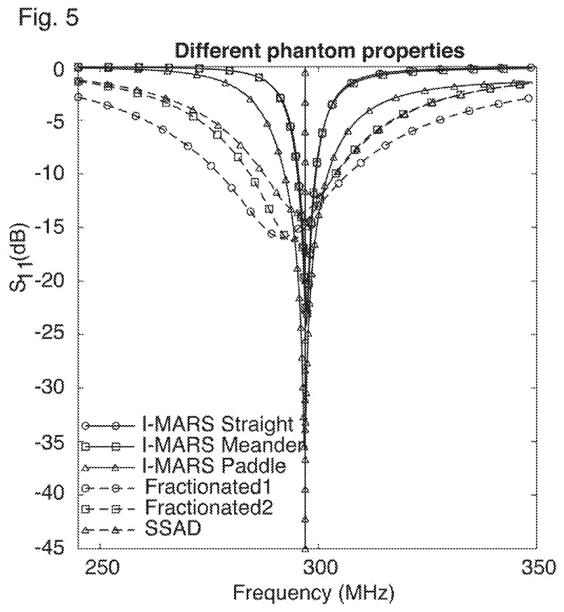

As shown in FIGS. 4A to 4D, all the I-MARS elements are significantly more robust to the change of distance to load, with a maximum detuning of a few MHz, while the conventional designs were detuned by up to 50 MHz. The I-MARS Straight and I-MARS Meander had better performance than the other designs, while I-MARS Paddle has slightly lower stability against loading variations than I-MARS meander.

Among the three conventional dipole elements, the SSAD antenna had the best performance, which is however noticeably inferior to the I-MARS designs. It is worth noting that in addition to better S₁₁, the I-MARS elements had a smaller bandwidth compared with existing dipole coil designs, potentially leading to improved signal-to-noise ratios.

Another aspect of loading variation is the change in load electrical properties, mimicking change in body composition between patients. This was investigated by simulating the coil elements at a distance of 10 mm from a phantom with relative permittivity of 38.5 and conductivity of 0.46 S/m. After achieving a S₁₁=−20 dB, the simulations were repeated with a different set of electrical properties of the phantom (relative permittivity of 71.5 and conductivity of 0.86 S/m), while using identical matching and tuning circuits.

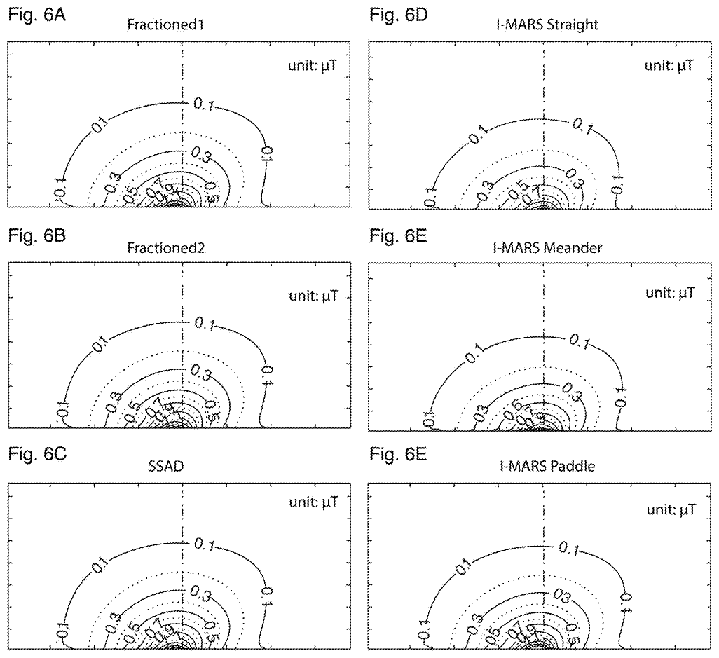

FIG. 5 shows that a frequency shift and decrease in matching was only observed in conventional elements, as I-MARS elements showed no frequency shift or degraded matching in this case. Such stability to tissue electrical properties would enable I-MARS elements to be used to scan a wide range of body parts with no performance degradation.

B₁ Power and SAR_{10g} Efficiency

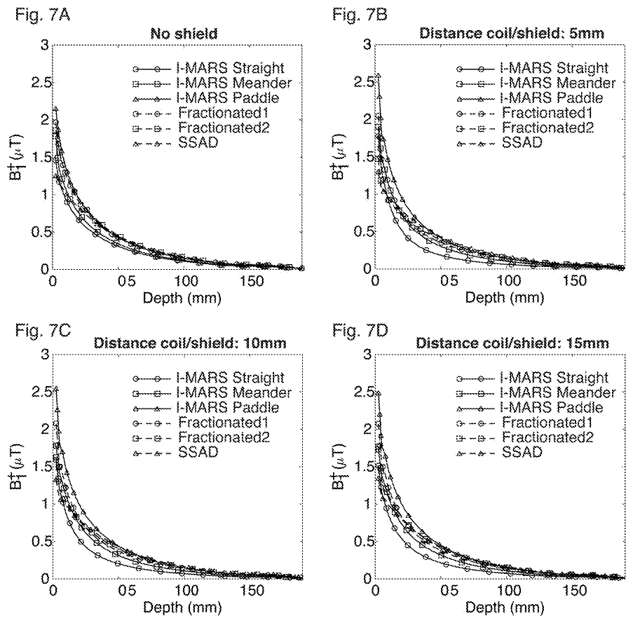

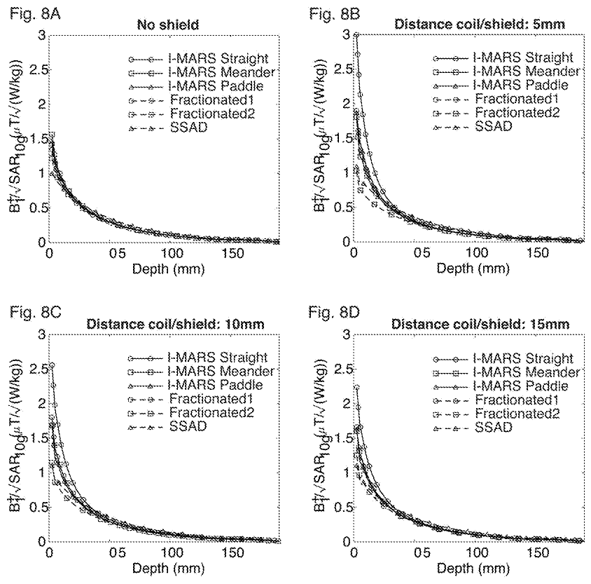

As shown in FIGS. 6A to 6F, the B₁⁺ fields of the six above mentioned antennas are compared on the central axial slice of the phantom with 1W accepted transmit power. The overall transmit profiles of all the elements are similar. To compare the B₁⁺ field penetration into the load, the B₁⁺ field magnitudes against distance from the surface of the phantom (along the dash lines in FIGS. 6A to 6F) are investigated, with no RF shield, as shown in FIG. 7A, and with an RF shield that is 5 mm, 10 mm and 15 mm away from the feed side of the dipoles/antenna, as shown in FIGS. 7B, 7C and 7D, respectively. The width and length of the shields were 20 mm larger than the maximum width and length of respective elements. FIGS. 7A to 7D show that the I-MARS Straight has a lower B₁⁺ strength per 1W accepted power than other designs (possibly due to its 380 mm length), while the I-MARS Paddle has the best overall efficiency as it was optimized for this purpose. Furthermore, the shields reduced the efficiency of the conventional designs, but had no negative effect on the power efficiency of the I-MARS designs.

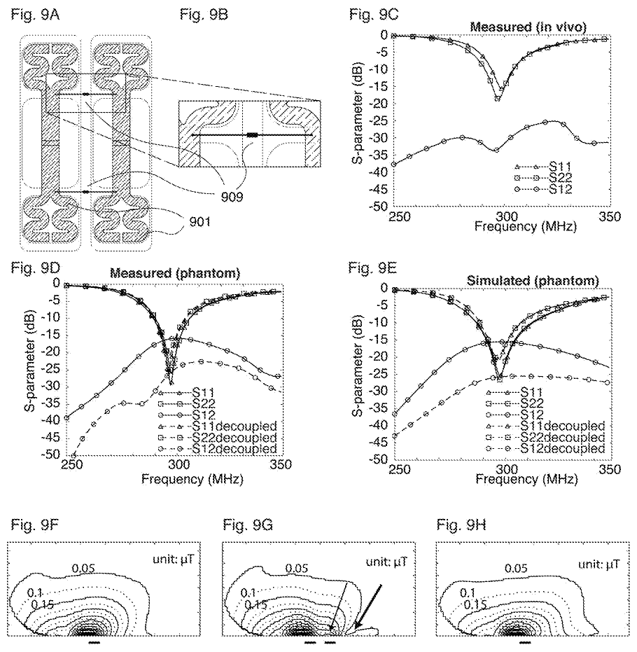

FIGS. 8A to 8D shows the same data normalized to the maximum SAR_{10g} in the phantom. All the I-MARS elements perform as well as or better than the conventional designs for any shield distance, with the I-MARS Straight having the best performance.

### Coupling of Various Coil Elements

To characterize the coupling between individual elements of an array, the scattering parameters of two elements of the same type were modelled, when they were located at varied distances. Simulation setup was similar to that of the previous study concerning changing loading conditions. Here, a second element of the same type was brought to the vicinity of the first element with a center-to-center distance of 120 mm, 80 mm, 70 mm and 55 mm. In all individual simulations, the two elements were tuned at 297 MHz and matched at −20 dB.

Table 3 shows the transmission coefficient S₁₂ when varying the inter-element distance between a pair of dipole elements of the same type. Among the conventional elements, the Fractionated2 element had the best decoupling performance. In comparison, all I-MARS based designed out-performed the conventional designs. The I-MARS Meander had the best isolation at larger distances (80 mm and 120 mm), with a S₁₂ 3 dB lower than that of the Fractionated2 dipole. The I-MARS Straight had the lowest coupling with short distances (55 mm and 70 mm), with S₁₂˜2.5 dB lower than the Fractionated2 dipole. The I-MARS Paddle behaved as well the Fractionated2 dipole at all inter-element distances, while having a more practical element dimension. Overall, the results indicate that the I-MARS coils have intrinsically high isolation between like elements, when the inter-element distance is varied in loaded conditions.

TABLE 3

| | Distance | Distance | Distance | Distance |
|---|---|---|---|---|
| | 120 mm | 80 mm | 70 mm | 55 mm |
| Fractionated1 (dB) | −23.3 | −15.4 | −13.2 | N/A |
| Fractionated2 (dB) | −25 | −17 | −14.8 | −11.5 |
| SSAD (dB) | −14.7 | −10 | −9 | −5.9 |
| I-MARS Straight (dB) | −27 | −20 | −17.3 | −13.8 |
| I-MARS Meander (dB) | −28 | −20 | −14.8 | N/A |
| I-MARS Paddle (dB) | −25.5 | −17.6 | −15.6 | −12.2 |

Compatibility of I-MARS with Existing Decoupling Methods

Although the I-MARS elements possess intrinsically high self-isolation facilitating dense coil arrays, even higher levels of decoupling would be desirable. In particular, the use of pTx techniques and receive performance of RF coils greatly benefit from lower coupling, to increase the degrees of freedom in transmission and reduce noise correlation, respectively. At lower fields, loop elements are typically used in local surface array coils, which can be decoupled using a variety of techniques, including:
overlapping, where the mutual inductance is cancelled by the flux passing through the overlapping area;
capacitive/inductive, where decoupling capacitors connect loops and form current pathways that cancel mutual coupling;
transformers, where mutual coupling between inductors placed in series with the coils cancel the coupling between loop elements.

However, these techniques cannot be directly applied to dipole elements, as loop antennas couple magnetically whereas dipole antennas mostly couple electrically, which is more challenging to mitigate. The use of passive elements and new amplifier designs were investigated to decouple dipole antennas, but was shown to affect the field distribution, add bulk and minimal distance constraints between neighbor elements, and may limit bandwidth and power efficiency.

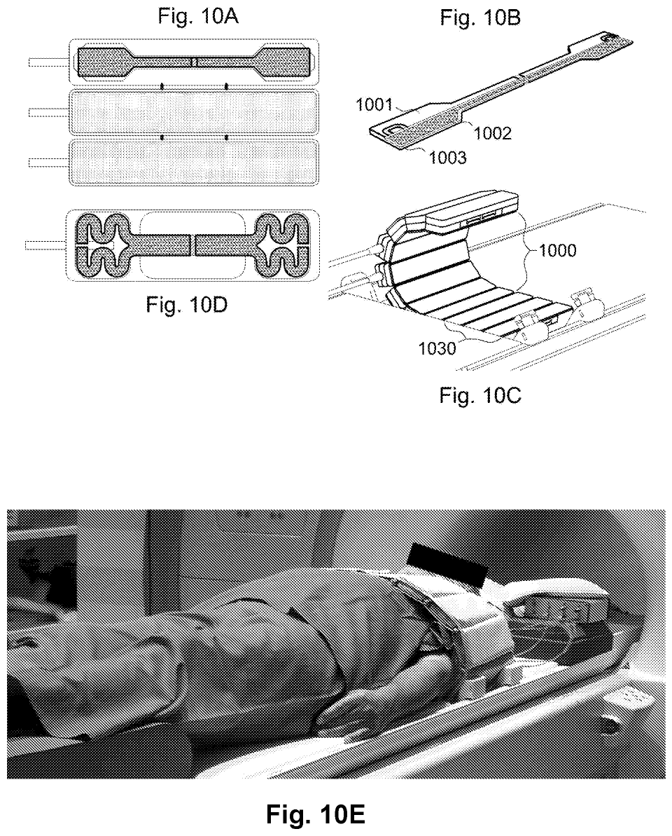

The unique design of I-MARS elements enables decoupling techniques between two I-MARS elements. In particular, it has been identified that inter-element isolation can be improved using inductive decoupling, by directly connecting multi-modal antennas 901 with inductors 909, as shown for example in FIGS. 9A and 9B.

As a proof of concept, when two I-MARS Meander elements with a center-to-center distance of 95 mm were connected with an inductor of 220 nH, decoupling of S₁₂=−35 dB was achieved at 297 MHz when loaded with a torso as shown in FIG. 9C. This level of isolation was stable when positioning the elements on different body parts, and is therefore compatible with a flexible coil. FIGS. 9D and 9E show similar experimental and simulated results, respectively, when loaded with a large phantom (εᵣ=74, σ=0.66 S/m). In this case, the decoupling was improved from S₁₂=−16 dB to −26 dB.

The effect of the decoupling network on the B i-field was simulated using two I-MARS Meanders (with RF shields) with a center-to-center distance of 70 mm. The ‘Perfect decoupling’ result shown in FIG. 9F, which serves as a baseline for comparison, was obtained by only simulating one channel. The ‘Without decoupling’ and ‘With additional decoupling’ cases shown in FIGS. 9G and 9H, were obtained by simulating the two channels without and with inductive decoupling inductors of 145 nH, respectively. In this case, the inductive decoupling improved S₁₂ from −13 dB to −31 dB.

The calculated B₁⁺ fields are shown in FIGS. 9F to 9H, while only one channel is driven with 1W of accepted power. With the ‘Without decoupling’ case in FIG. 9G, the small and large arrows indicate the coupling-induced constructive and destructive interferences of the Bit, respectively, which was absent in the ‘With additional decoupling’ case shown in FIG. 9H. The channels in the ‘With additional decoupling’ case operate virtually independently of each other, potentially providing improved pTx efficiency and SNR performance in local arrays coils.

### Prototyping and In Vivo Imaging Tests

To verify the performance predicted by numerical simulations, arrays of I-MARS elements were manufactured using the FR-4 process, with R04360G2 laminates (Rogers Corporation, Chandler, Ariz., USA). The low-loss substrate R04360G2 was adopted owing to its high relative permittivity of 6.15 and low electrical loss (Dissipation Factor 0.0038 at 10 GHz/23° C.).

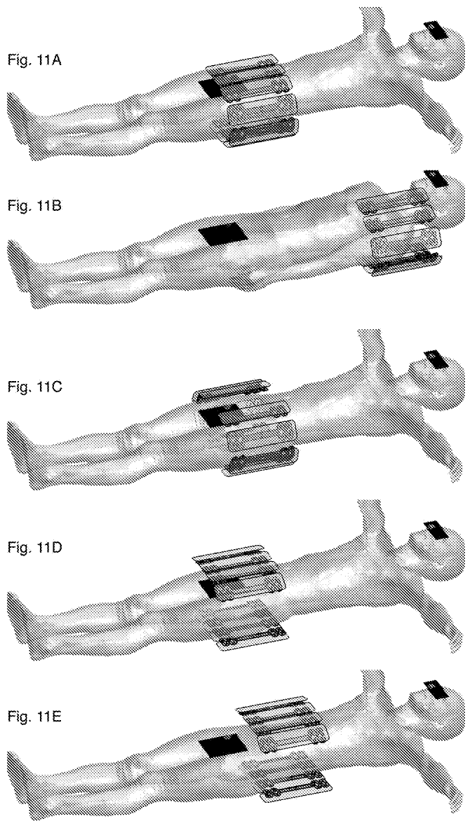

As an example, manufactured I-MARS Paddle elements with their 3D printed PETG housings are shown in FIG. 10A. FIG. 10B shows computer-aided models of an I-MARS Paddle element, including an outer conductive dipole element 1001, an internal conductive element 1002 and a dielectric material 1003. FIG. 10C shows a computer-aided model of an assembled eight-channel coil array, including active antennas 1000 and dummy padded blocks 1030.

FIG. 10D shows a manufactured I-MARS element in an acrylic housing, which was used for unilateral shoulder imaging of a volunteer using the array of FIG. 10E. For this coil, each RF antenna and RF shield were fully enclosed in acrylic formers and self-contained, and can be expanded into different array configurations in a straightforward fashion. Individualized RF shields were attached to the housing at 13 mm away from the antenna on the feed side. As shown in FIG. 10E, such elements were combined into an array, allowing the relative position of the elements to be adjusted to facilitate imaging of various body sections. A balanced feeding mechanism is used to drive the coils, enabling symmetric current flow and distributed electric fields.

The I-MARS antennas are robust to loading changes and have high isolation between neighboring elements. In the presented configurations, additional inductive decoupling was not necessary or implemented, thanks to sufficient decoupling provided by the required distance between elements. Since retuning and/or re-matching of RF antennas are uncommon for in vivo MR imaging, these features make possible imaging of different body sections without compromising transmit and receive performance. UHF imaging will also benefit from the high B₁⁺ efficiency against RF power and SAR provided by the I-MARS. Here, the constructed 8-element I-MARS Meander coil array prototype was employed for imaging healthy volunteers of various body sections, including unilateral hip, unilateral shoulder, bilateral hip, prostate and lumbar spine. Across these five imaging scenarios, the geometric configurations of the I-MARS Meander array were readily adjusted to provide the best conformity, as illustrated in FIGS. 11A to 11E.

For unilateral hip and shoulder imaging, as illustrated in FIGS. 11A and 11B, I-MARS elements were arranged in C-shape to conform to the left hip and shoulder of the subject, respectively. To reduce the unnecessary field of view, the elements were placed next to each other with a center-to-center distance between next neighbors of 80 mm, and by using only six channels in the case of shoulder imaging.

As shown in FIG. 11C, the 8 elements of the array were distributed around the lower abdominal section of the body for bilateral hip imaging. The center-to-center distances between elements were between 110 and 184 mm. For lumbar spine and prostate imaging shown in FIGS. 11E and 11D respectively, the elements of the array were arranged into anterior and posterior groups, each of which consists of four elements with 80 mm center-to-center distance between next neighbors. As demonstrated, the elements were subjected to varied loading conditions due to tissue composition and conformity of body parts; additionally, the distances between elements were required to change to best accommodate anatomy in different imaging scenarios.

Numerical electromagnetic simulations using a finite-difference time-domain (FDTD) method have been performed for each of the imaging scenarios. The simulations were assisted by software package Sim4Life (ZMT, Zurich, Switzerland) with digital human models, as shown in FIGS. 11A to 11E. A set of virtual observation points (VOPs) for each body section were calculated using a procedure similar to that previously reported (Jin J, Weber E, Destruel A, O'Brien K, Henin B, Engstrom C, Crozier S “An open 8-channel parallel transmission coil for static and dynamic 7T MRI of the knee and ankle joints at multiple postures”. Magnetic Resonance in Medicine 2017) and applied for in vivo imaging to ensure subject safety against RF exposure. A safety factor of two was implemented for an RF safety margin. All scans were performed well within the SAR limits enforced by IEC. For each body region, the imaging protocol consisted of a global B₀ shim, a localized B₁ shim over the region of interest, a 3D water-excited Dual-Echo Steady-State (we-DESS) and Turbo Spin Echo (TSE). The acquisition times of the we-DESS were less than 6 minutes to maintain patient comfort and minimize motion-related artefacts. The parameters of the we-DESS for each region are listed in Table 4.

TABLE 4

| | TA | Data | Resolution | FOV | Flip | BW | TE/TR | | |
|---|---|---|---|---|---|---|---|---|---|
| 3D-DESS | (min:sec) | Matrix | (mm) | (mm) | Angle | (Hz/Pixel) | (msec) | Grappa | Measurements |
| Unilateral | 5:23 | 540 × 640 | 0.56 iso | 303 × 360 | 25 | 425 | 3.1/11 | 3 | 1 |
| Hip | | | | | | | | | |
| Unilateral | 4:05 | 264 × 384 | 0.7 iso | 185 × 270 | 20 | 250 | 3.77/12.2 | 2 | 2 |
| Shoulder | | | | | | | | | |
| Bilateral | 4:43 | 512 | 0.7 iso | 360 × 360 | 25 | 425 | 3.1/11 | 3 | 1 |
| Hip | | | | | | | | | |
| Spine | 5:47 | 448 | 0.67 iso | 300 × 300 | 25 | 429 | 3.1/11 | 2 | 1 |
| Prostate | 3:38 × 2 | 704 × 704 | 0.3 × 0.3 × 3 | 210 × 210 | 180 | 273 | 94/7000 | 2 | 2 |
| (T2w-TSE) | | | | | | | | | |

Images were acquired on different healthy volunteers using the prototype I-MARS coil array:
Left hip joint: Male, 53 years, 78 kg
Bilateral hip joints: Female, 26 years, 65 kg
Left shoulder joint: Male, 34 years, 81 kg
Prostate: Male, 29 years, 83 kg
Lumbar spine: Male, 53 years, 78 kg

Images were acquired on a prototype whole-body 7T MR research scanner (Siemens Healthcare, Erlangen, Germany). A custom 8-channel transmit/receive switch was employed to interface the I-MARS coil array to the MR scanner. The medical research ethics committee of the University of Queensland approved the current study, and informed written consent was obtained from all participants who had no history of significant musculoskeletal pathology.

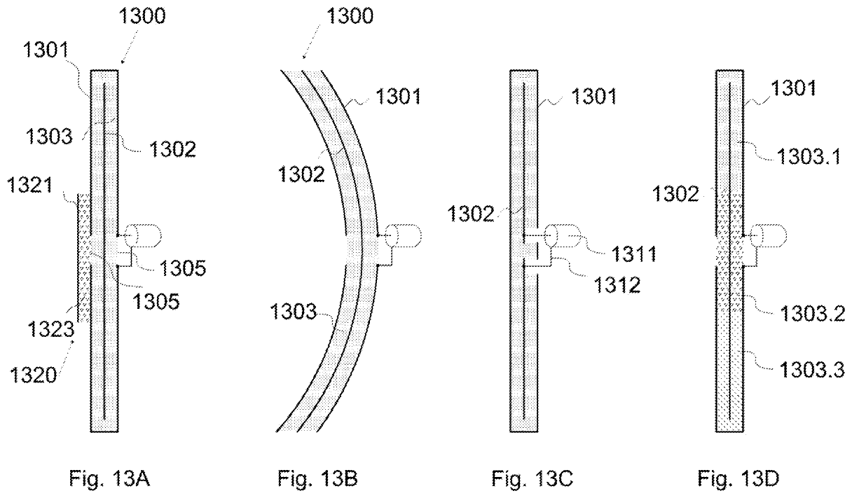

Coronal unilateral and bilateral hip DESS images (0.56 and 0.7 mm isotropic resolution without interpolation) are shown in FIGS. 12A and 12B, respectively. A custom B₁ shimming and SAR control algorithm was used to calculate uniform RF excitation fields over the regions of interest (ROI), indicated by the dashed white lines. Signal dropouts (large arrows) can be observed over the inner thighs of the bilateral scan, but these do not affect the diagnostic value in the images.

FIG. 12C shows an axial view of the shoulder joint acquired with DESS (0.7 mm isotropic resolution), with uniform B₁ across the field-of-view achieved with six I-MARS antennas. FIG. 12D shows a coronal T2w-TSE of the prostate (0.3 mm in-plane resolution, 3 mm slice thickness). Signal dropout was observed in the bladder, but it did not affect the ROI near the prostate.

Sagittal views of the lumbar spine we-DESS are shown with 0.67 mm isotropic resolution without interpolation on the same volunteer, when performed using four posterior elements only in FIG. 12E, then additionally using four anterior elements FIG. 12F. Artifacts due to respiration and bowel motion are visible in the latter case, but do not noticeably affect the image quality over the lumbar spine. The B₁ shimming provided uniform RF excitation fields over the lumbar spine and was more effective when all 8 elements were utilized as shown in FIG. 12F.

### Variations

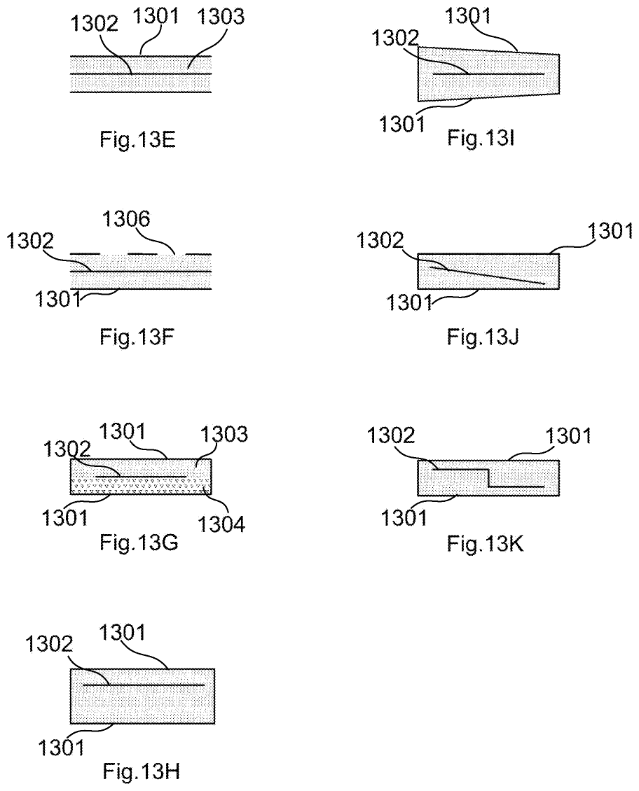

It will be appreciated from the above that a wide range of different configurations could be implemented that allow for the combined dipole and transmission-line modes of operation, which in turn lead to a number of the benefits previously outlined. A number of these variations are shown in FIGS. 13A to 13K, which use similar reference numbers to FIGS. 1A to 1D, albeit increased by 1200.

For example, the I-MARS antennas could include additional secondary elements, such as capacitive elements to adjust properties of the antenna, for example to perform tuning for specific applications. An example of this is shown in FIG. 13A, in which an antenna similar to that of Configuration D of FIG. 1D is modified by the addition of a capacitive element.

In this example, the antenna 1300 includes an outer conductive element 1301 in the form of a dipole including a slot 1305 and an inner conductive element 1302 contained within a dielectric layer 1303. The secondary element additional layer includes an outer secondary conductive element 1321 and a dielectric layer 1323 extending across the slot in one side of the antenna, which can alter coupling between arms of the dipole.

In the example of an antenna element shown in FIG. 13B (sagital cross-section view) and 13E (axial cross-section view), an antenna similar to that of Configuration D of FIG. 1D is provided including three conductive elements 1301, 1302, 1304. In this example, the antenna is curved so that the antenna can conform to a shape of the subject, to thereby optimize the field generated within the subject. Additionally, in this example, the outer conductive elements 1301, 1304 cover the outer faces of the dipole and therefore have similar dimensions to the inner conductive element 1302.

FIG. 13C is a schematic cross-sectional view of an I-MARS antenna when the inner conductive element 1302 is primarily coupled to the RF system 1311, so that the outer conductive element 1301 is stimulated by the inner conductive element.

FIG. 13D is a schematic cross-sectional view of an I-MARS antenna, including first, second and third dielectric materials 1303.1, 1303.2, 1303.3 having different properties, distributed along a longitudinal of the element.

FIGS. 13F to 13K are schematic axial cross-sectional views of I-MARS antenna of FIG. 1D, including a number of other alterations, including two slots 1306 in a front conductive element (FIG. 13F); a second dielectric material 1304 having different properties (FIG. 13G); an inner conductive element 1302 parallel to but asymmetrically placed with respect to outer conductive elements 1301 (FIG. 13H); an asymmetric outer geometry with axially converging outer conductive elements 1301 (FIG. 13I); an axially sloped inner conductive element (FIG. 13J); and an axially stepped inner conductive element (FIG. 13K).

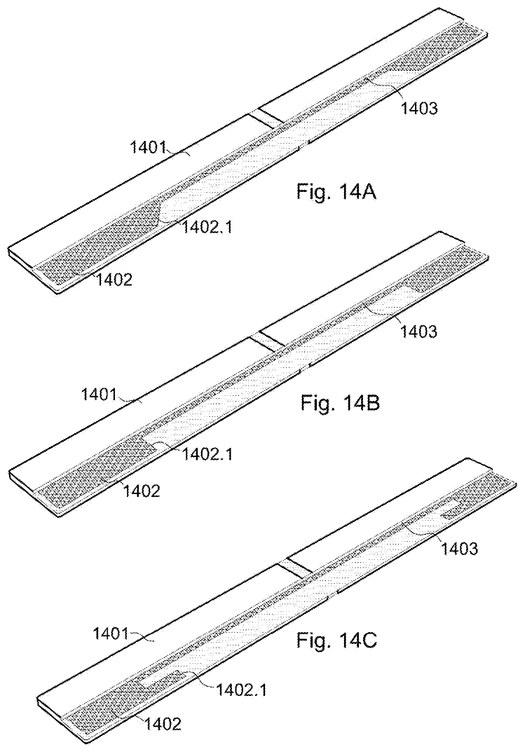

FIG. 14A to 14C illustrates additional variations of I-MARS antennas in the three-quarter sectional view, in a similar fashion to the illustration of paddle antenna shown in FIG. 10B.

In these examples, the inner and outer conductive elements of the I-MARS Paddles 1401, 1402 are largely of the same shape as in the example of FIG. 10B. However, in these examples, the outer conductive element 1401 and dielectric layer 1403 are of a different shape (primarily rectangular in these examples) compared to the inner conductive element 1402. In these examples, it is also shown that geometric variations can be made to the inner conductive element, while the dielectric layer and outer conductor remain unchanged. For example, the design of the inner conductive layer 1402 of FIG. 14B is similar to that of FIG. 14A, albeit with a chamfer 1402.1 of the paddle having an angle of approximately 90°, as opposed to 45° in the case of FIG. 14A. Similarly, the design of the inner conductive layer 1402 of FIG. 14C is based on the arrangement of FIG. 14B with two symmetrical extrusions 1402.2 extending from the chamfer 1402.1 along the longitudinal direction of the I-MARS antenna towards its middle portion.

It will be appreciated that the overlap between the two the conductive elements, together with the dielectric between them, determines the characteristics of the antenna, and so variations to the inner conductive element can be used to alter the characteristics of the resulting antenna, without altering the appearance of the antenna. However, conversely, the size and/or shape of the outer conductive element could be altered, whilst the inner conductive element remains unchanged to also alter the characteristics of the antenna.

### CONCLUSION

The simulation results and acquired images presented in this work suggest that the proposed Integrated Multi-modal Antenna with coupled Radiating Structures (I-MARS) elements provide advantages for UHF MR imaging. Compared with the state-of-the-art dipole coil elements, the individual I-MARS has high efficiency in terms of producing transmit magnetic fields normalized to accepted power and normalized to peak SAR_{10g}; demonstrating superior stability against loading changes; and presenting intrinsically higher isolation between neighboring elements when the distance between elements changes significantly. Furthermore, I-MARS elements are compatible with decoupling techniques that provide better than −25 dB isolation in the tested configurations. This combination of advantages is unique to I-MARS, making a multi-element I-MARS array uniquely suitable for multi-anatomy UHF imaging, where array elements can be rearranged to accommodate different body parts without the need for additional adjustments of tuning, matching and decoupling, and without sacrificing coil performance.

This work aims to provide an RF coil-element design addressing all four of the aforementioned design criteria, making it ideally suited for RF transmission and/or reception for ultra-high field MRI/MRS. The proposed coil-element has low sensitivity to loading changes; provides superior inter-element isolation (when part of a coil array), and a better efficiency regarding RF energy deposition. These benefits enable imaging versatility, allowing the proposed antenna or an array of such antennas to be re-arranged for imaging various body parts with optimal performance in all configurations. Namely, the elements can be arranged to conform to the body shapes and body parts, while varied inter-element distance and varied body composition will not introduce a notable loss of efficiency.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

1. A multi-modal antenna for use in magnetic resonance applications, the multi-modal antenna including:
a) an elongate first conductive element;
b) an elongate second conductive element at least partially aligned with and spaced from the first conductive element; and,
c) a dielectric material at least partially separating the first and second conducting elements so that the first and second conductive elements are electromagnetically coupled and/or electrically connected, and wherein at least one of the first and second conducting elements are configured to be electromagnetically coupled and/or electrically connected to an RF system so that the multi-modal antenna can at least one of transmit and receive RF electromagnetic signals for performing magnetic resonance imaging or spectroscopy.

2. A multi-modal antenna according to claim 1, wherein at least one of:
a) the first and second conducting elements operate in one of:
i) a transmission line mode;
ii) a dipole mode; and,
iii) a combination of a transmission line mode and a dipole mode; and,
b) the dielectric layer and the first and second conductive elements form a transmission line.

3. A multi-modal antenna according to claim 1, wherein at least one of:
a) the first conductive element is stimulated by the RF system and the second conductive element is stimulated by the first conductive element; and,
b) the first and second coupled conductive elements are stimulated by the MR signal from the subject.

4. (canceled)

5. A multi-modal antenna according to claim 1, wherein the first and second conductive elements cooperate to define a closed-loop current including conductive currents passing along the first and second conductive elements and displacement currents passing through the dielectric material.

6. A multi-modal antenna according to claim 1, wherein at least one of the conductive elements has a dipole configuration.

7. A multi-modal antenna according to claim 1, wherein at least one of:
a) at least one of the conductive elements includes a slot or cut-out to define two arms, and wherein the RF system is electrically connected and/or electromagnetically coupled to each arm; and
b) each conductive element at least one of:
i) includes slots or cut-outs;
ii) has a length greater than a width;
iii) has a width greater than a thickness;
iv) is substantially laminar;
v) is substantially planar;
vi) is at least partially flexible so that the multi-modal antenna can conform to a shape of a subject;
vii) is at least partially curved so that the multi-modal antenna can conform to a shape of a subject;
viii) includes an axial cross sectional shape that is at least one of:
(1) rectangular;
(2) circular; and,
(3) elliptical;
ix) has a paddle-shaped profile including one or more end portions wider or narrower than a mid-portion;
x) has one or more meandering portions extending widthwise and lengthwise to increase an effective electrical length of the conductive element;
xi) includes multiple paddle stages;
xii) includes multiple paddle stages having different relative widths; and,
xiii) includes multiple stages having different relative widths, and wherein a chamfer angle between stages can be adjusted.

8. (canceled)

9. A multi-modal antenna according to claim 1, wherein the first and second conductive elements are interconnected via at least one of:
a) lumped elements;
b) additional conductive elements; and,
c) a direct connection.

10. A multi-modal antenna according to claim 1, wherein the second conductive element at least one of:
a) is smaller than the first conductive element;
b) is shorter than the first conductive element;
c) is narrower than the first conductive element; and,
d) has a complementary profile to the first conductive element.

11. A multi-modal antenna according to claim 1, wherein at least one of:
i) a spacing between the first and second conductive elements is at least one of:
iii) at least 0.1 mm;
iii) at least 1 mm;
iv) less than 10 mm; and,
v) about 3 mm; and,
b) the first and second conductive elements are spaced at least one of:
i) in a substantially parallel arrangement; and,
ii) asymmetrically.

12. (canceled)

13. A multi-modal antenna according to claim 1, wherein the dielectric material is at least one of:
a) is partially sandwiched between the first and second conductive elements;
b) is provided in a layer;
c) includes a number of layers of dielectric material; and,
d) includes at least two different materials having different dielectric properties.

14. A multi-modal antenna according to claim 1, wherein the multi-modal antenna includes:
a) a dielectric layer;
b) an outer conductive layer on at least one surface of the dielectric layer; and
c) an inner conductive layer within the dielectric layer.

15. A multi-modal antenna according to claim 1, wherein at least one of:
a) the outer conductive layer includes the first conductive element; and an inner conductive layer includes the second conductive element; and,
b) the dielectric material has a permittivity constant of at least one of:
i) at least 1;
ii) less than 10;
iii) less than 35;
iv) less than 50;
v) less than 100;
vi) less than 250;
vii) less than 500;
viii) less than 1000; and,
ix) about 3.5.

16. (canceled)

17. A multi-modal antenna according to claim 1, wherein at least one of:
a) the antenna includes at least one further conductive element and/or at least one further dielectric structure; and
b) the antenna includes at least one secondary element that modifies an electromagnetic response of the antenna.

18. (canceled)

19. A multi-modal antenna according to claim 1, wherein the antenna includes at least one secondary element that modifies an electromagnetic response of the antenna and wherein at least one of:
a) the at least one secondary element includes at least one of:
i) at least one secondary dielectric material; and,
ii) at least one a secondary conductive element; and
b) the at least one secondary element spans a cut-out in the first conductive element.

20. (canceled)

21. (canceled)

22. A multi-modal antenna according to claim 1, wherein the multi-modal antenna includes a housing configured to maintain a desired spacing between the subject and the first and second conductive elements.

23. A multi-modal antenna according to claim 22, wherein the housing includes a foam for engaging the subject, the foam having a defined thickness to maintain the desired spacing.

24. A multi-modal antenna according to claim 1, wherein the RF system includes at least one of:
a) a signal generator configured to generate RF signals that are applied to the antenna to generate the RF electromagnetic field;
b) a detector that detects signals originating within the subject; and,
c) a control system that causes the RF system to send control signals to control supporting electronics including at least one of:
i) active detuning circuits;
ii) switching electronics; and,
iii) active switches.

25. A multi-modal antenna according to claim 1, wherein active switching electronics are implemented into the multi-modal antenna to enable at least one of:
a) active detuning to allow separate transmit and receive antenna operation modes;
b) active on/off switching of different segments in conductive elements to allow control of current and field distributions;
c) active changing of the resonant frequency; and,
d) active changing of the effective electrical length of the multi-modal antenna.

26. A multi-modal antenna array for use in magnetic resonance applications, the multi-modal antenna array including a plurality of RF antennas, each RF antenna including:
a) an elongate first conductive element;
b) an elongate second conductive element at least partially aligned with and spaced from the first conductive element; and,
c) a dielectric material at least partially separating the first and second conducting elements, wherein the first and second conductive elements are electromagnetically coupled and/or electrically connected, and wherein at least one of the first and second conducting elements are configured to be electromagnetically coupled and/or electrically connected to a multi-modal system so that the RF antenna can at least one of transmit and receive RF electromagnetic signals for performing magnetic resonance imaging or spectroscopy.

27. A multi-modal antenna array according to claim 26, wherein at least one of:
a) the antenna array includes additional decoupling technique between the antennas in the array; and,
b) active detuning is implemented to allow separate transmit and receive antenna array configurations.

28. (canceled)

29. (canceled)
